(19) United States
(12) Patent Application Publication
(10) Pub. No.: US 2006/0155214 A1
(43) Pub. Date: Jul. 13, 2006

(54) Urine collection devices and urine collection systems

(76) Inventor: Paul Wightman, Oxford (GB)

Correspondence Address: YOUNG & THOMPSON, 745 SOUTH 23RD STREET, 2ND FLOOR, ARLINGTON, VA, 22202, US

(21) Appl. No.: 10/525,757

(22) PCT Filed: Aug. 28, 2003
(86) PCT No.: PCT/GB03/03788

§ 371 Date: Sep. 26, 2005

(30) Foreign Application Priority Data
Aug. 28, 2002 (GB) 0219944.6
Apr. 11, 2003 (GB) 0308442.9
Apr. 14, 2003 (GB) 0308553.7

(51) Int. Cl. A61B 5/00; B65D 81/00

(52) U.S. Cl. 600/574

(57) ABSTRACT

A urine collection device for attachment to a pump and for a user in a supine, or reclined position, includes: an open-mouthed container having a rim around the mouth for being urged in use to seat generally around the periphery of the urine discharge region to receive urine discharged from the urethra, the container being adapted to fit between the legs of a user in a supine or reclined position, the container having at its lower part a reservoir basin in which fluid may collect in use when the container is applied to a user in a supine or reclined position, and an outlet in the lower part of the reservoir basin through which urine may be drawn from the container.

This invention relates to a urine collection device and urine collection systems. In particular and not exclusively, the urine collection device is configured such that it can be connected to a negative air pressure, such as from a suction pump to allow collection of urine when the user is in a number of positions, including both the supine and reclining positions.

Devices for the collection of urine from female users who are bedridden are known. Such devices often involve catheters that have to be inserted within the urethra of the user, which in itself can be an uncomfortable procedure. Further, catheters often form a site where bacteria can accumulate which may result in urinary tract infections and possibly bladder infections thereby leading to complications in the treatment of an individual.

It is known to use specially shaped funnels, which are shaped to the contours of the female genital region and these collect and conduct urine away from the user's body. Such urinary funnels are disclosed in EP 464575 and WO 90/13280 and GB 2362577. However, such known funnels are not convenient for use in the reclined or supine positions, because they do not form an adequate seal between the user, and also “puddling” of urine within the device can occur; that is urine may collect in a space between the funnel and the user's body which does not drain, and which therefore leaks as soon as the funnel is removed. Consequently, known urine collection devices are prone to leakage with the result that both the user and bed linen may become soiled by leaked urine. Further, known devices are not designed for use when the user is in a number of positions, for example lying down, (i.e. supine) or sitting in a reclining position.

In this specification, the terms “upper”, “lower”, “top” and “bottom” refer to a collection device when positioned in use on a user, ready to collect urine.

Likewise, the terms “front”, “back” and “forward”, “rearward” refer to the device when viewed in use with “front” and “forward” being in the direction towards the feet of the user and “back” and “rearward” being towards the head of the user.

An aim of the present invention is to provide a hygienic and simple to use urine collection device that reduces the possibility of leakage of urine from the device, and which can be used when the user is in a number of positions.

According to one aspect of this invention, there is provided a urine collection device for attachment to a pump and for a user in a supine or reclined position, said device including:

an open-mouthed container having a rim around the mouth for being urged in use to seat generally around the periphery of the urine discharge region to receive urine discharged from the urethra,

the container being adapted to fit between the legs of a user in a supine or reclined position,

the container having at its lower part a reservoir basin in which fluid may collect in use when the container is applied to a user in a supine or reclined position, and

an outlet in the lower part of the reservoir basin through which urine may be drawn from the container.

Preferably said rim is generally of oblate curvilinear form wherein a broader, upper end region of the rim is adapted to be seated against the mons pubis (pubic bone region), and a narrower lower end region of the rim is adapted to be seated against the perineum, in use. The lower end region of the rim is advantageously adapted to engage edgewise into the perineum of the user when the urine collection device is urged into a urine-collecting position in use, to effect a fluid tight seal. The urine collection device preferably includes a forwardly facing lobe, knob or protrusion designed to guide the hand of a user such as to press the lower end region of the rim edgewise into the perineum in use. The inner surface region of the container extending inwardly from the lower end region of the rim advantageously defines a downwardly-inclined weir surface to ensure that urine does not pool at the interface.

The upper and lower ends of the rim preferably define a notional contact plane (xx) which in use lies at between about 105° to 145° to the horizontal when the user is reclined or supine. The reservoir basin is defined at least partly by a back wall portion and a bottom wall portion which converge downwardly towards said outlet.

The reservoir basin has a capacity of at least 10 ml and preferably at least 20 ml, when the device is applied to a user in a supine position.

The back wall portion and said bottom wall portion conveniently converge at an angle of between 90° and 120°, and preferably at about 106°. The back wall portion preferably lies at an angle of between about 120° and 160°, and ideally at about 146° to the notional contact plane.

The urine collection device may further include in the lower region of the mouth at least one weir plate means which extends between opposed portions of the rim and extends into the basin region of the container.

The outlet may include a tubular portion integral with the container and having a drain inlet end which is located at the lowermost portion of the reservoir basin, and which extends upwardly to terminate in a drain outlet end.

The rim preferably has an inturned resiliently deformable lip extending around a part thereof for seating against the user's body and providing an internal peripheral gutter.

The device advantageously includes a vent aperture for air, which preferably is in the form of a generally vertical breather tube to prevent escape of urine, and whose other end is protected from inadvertent blocking. The size of the vent may be selected having regard to the suction applied to the device in use so as to maintain a slight negative pressure when the device is applied to the user's body.

The invention extends to a urine collection system incorporating a urine collection device as described above in combination with a suction pump and a container for receiving urine. The system may include a urine sensor associated with said urine collection device which is used by the system to automatically turn on the pump as soon as urination begins.

The illustrated embodiments of the invention provide a urine collection device comprising a funnel having an upper wall region positionable towards the pubic bone area of the user, side walls being positionable either side of the area of the user's body from which urine is expelled, and a bottom wall region, positionable against the perineum of the user, the upper edge of the funnel forming an opening which extends around the area of the user's body from which urine is expelled, the funnel also having an outlet by which urine can be removed from the urine collection device, wherein the outlet comprises a tube having its inlet end at or near the lowest part of the inside of the funnel and its outlet end above the level of the inlet to thereby provide a urine reservoir in the lower part of the funnel, when the funnel is in use.

Embodiments of the invention also provide a urine collection device comprising a funnel having an upper wall region positionable towards the pubic bone area of the user, side walls being positionable either side of the area of the user's body from which urine is expelled, and a bottom wall region, positionable against the perineum of the user, the upper edge of the funnel forming an opening which extends around the area of the user's body from which urine is expelled, the funnel also having an outlet by which urine can be removed from the urine collection device, wherein the opening for the funnel leads to a urine receiving area formed by the front wall and forward parts of the side walls of the urine collection device, said urine receiving area leading to a urine collection reservoir formed by the bottom wall and rearward parts of the side walls of the urine collection device, said urine collection reservoir having a back wall extending from the opening of the funnel in the vicinity of the perineum of the user towards the bottom wall so that in use the urine collection reservoir defines with the adjacent area of the user's body a substantially closed chamber, which receives urine indirectly from the user via the urine receiving area, said urine collection reservoir leading to the outlet so that urine can be removed from the funnel.

In the illustrated embodiments, the opening of the funnel is contoured such that it generally has a concave shape when viewed from the side, with the upper part and the lower part of the opening projecting in comparison with the side walls of the opening. The upper and lower parts define a general medial axis of contact with the user's body, with the remaining parts of the circumference of the opening being in contact with the user's body.

It is desirable that at least part of the opening of the funnel includes an inwardly curling lip which forms a gutter that assists in directing urine towards the urine-collection reservoir.

Preferably, the lip extends around the whole periphery of the opening apart from that region which is adjacent the perineum thus providing a softer sealing surface around most of the periphery of the device, but a more inflexible cupping wall adjacent the perineum.

In the illustrated embodiments the inner surface of the device adjacent the area which in use cups against and into the perineum is arranged to form a downwardly inclined weir surface falling away from the line of contact with the perineum. By having this arrangement, the proportion of the urine generally running down that part of the user's genital area enclosed by the funnel region, i.e. urine which is not being propelled into the receiving area, is caused to be deflected back into the device that it is directed towards the collection reservoir and the outlet end, thereby avoiding leakage.

The device may include two separately formed parts, one of a softer material designed to engage the user's body with some give in the lip to allow deformation to form a good seat with the body of the user, and the other of a more rigid plastics material. Advantageously, the first part forms a detachable upper part of the device which attaches to the other part of more rigid material, such that where multiple devices are used in e.g. hospitals, respective upper and lower parts of the device can be interchangeable. This allows e.g. provision of lower parts with different capacities.

In a preferred arrangement, the back wall of the urine collection reservoir is sloped away from the medial axis of contact, in a direction away from the users body.

Advantageously, the back wall is at an angle of between 120° and 160° and more preferably at an angle of 130-150° from a plane formed by the two-point axis.

Ideally, the back wall is at an angle of about 146° from a plane formed by the two-point axis of contact.

It is preferred that the bottom wall of the urine collection device is at an angle of about 106° from the back wall of the device but this angle may vary by 15° either side of this value.

Advantageously, the outlet has an opening leading from the urine collection reservoir, which is positioned such that said opening is directed towards an area of the device where the back wall and the bottom wall of the urine collection reservoir meet. The outlet may be formed as a tube within the body of the device, with a wall of the tube merging into the bottom wall of the urine collection device.

It is also envisaged that there may be further weirs in proximity to the opening of the urine collection device, said weirs being in contact with the user's body so that urine may be more efficiently directed towards the inner surface of the funnel. The use of said weirs has the advantage that they assist in directing the flow of urine from the user into the body of the urine collection device.

Preferably, the weirs each have a curved surface. By having a curved surface, this assists in preventing the weirs from being deflected counter to the flow of urine due to the pressure of urine within the device. If deflection is avoided, there is a reduction in splashing of urine. This assists in encouraging cross flow of urine back into the body of the device and prevents any downward flow of urine from the device to the perineum and ultimately down the legs of the user and possibly onto the nursing staff.

It is preferred that the outlet for the funnel can be connected to a tube of about 8 millimetres internal diameter in order to drain urine from the funnel at a flow rate of up to 60 millilitres per second.

Preferably the capacity of the collection reservoir of the funnel is at least 20 millilitres when being used in a supine position to avoid the risk of leakage.

Advantageously, the inner surface of the front wall of the funnel, which forms the front part of the urine-receiving reservoir, has a smooth concave contour so that urine impinging thereon is deflected towards the urine collection reservoir. The concave surface acts as a deflector for a urine stream and so the flow is controlled such that it may be directed into the body of the reservoir. Also, by having a concave front face, there is the avoidance of turbulence, which could otherwise create a fluid/air mix that may reduce the effectiveness of drainage from the funnel, particularly when used with a suction pump.

Preferably the urine collection device has a hand locating area on the external walls of the device forming the urine receiving reservoir. The hand locating area may be provided by visual or tactile areas that the user can locate so that they know when they are holding the urine collection device in the most appropriate orientation for use, whereby the user's hand is directed to be applied in the correct direction so that the lower portion of the rim of the device is pulled edgewise into firm sealing engagement with the perineum into that area, thereby avoiding leakages.

It is envisaged that the funnel may include one or more air inlet apertures. Preferably, a single air aperture is provided in the back wall of the funnel immediately above the urine collection reservoir, the opening of the aperture leading to a tube extending through the back wall of the funnel in the urine receiving area in a generally upright direction and leading to an opening on the outside of the funnel. The opening is preferably surrounded by wall or other portions defining finger guards on the outside of the device so as to prevent accidental blockage of air into the device. The or each aperture may be in the walls of the part of the device forming the urine-receiving reservoir. The or each aperture may be valved, such as by means of a one-use foam valve. The valved arrangement may allow for pressure within the urine collection device to be regulated if urine is being removed from the device using a pump. In one arrangement the valves could be self-regulating, in that the valves are caused to operate when a certain negative pressure is reached within the device. The air inlet apertures allow air into the urine collection device before and during urination and this stops negative pressure from the pump building up, which may harm a user of the device. If required, in one embodiment, as urination occurs, (which may be detected by a sensor within the valve or the body of the device), the valve or valves are caused to close so that leakage from the device is prevented. Alternatively, the aperture may be of restricted cross section to maintain a slight negative pressure in the device in use.

The arrangement of the angles of the walls of the various parts of the urine collection part preferably have defined angles relative to one another and curved surfaces so that urine is caused to flow through the device at a good rate whilst avoiding excessive turbulence that could otherwise disrupt the flow dynamics of the urine within the device.

Further, by having an outlet that is offset from the opening of the funnel, urine flow is fed to the outlet in a controlled manner rather then being able to directly reach the outlet as in known devices. Urine instead collects at the lowest point of reservoir, thereby leading urine to the mouth of the outlet part so that urine can be sucked away. Further, the device can be used when the user is both in the sitting and supine positions. This is because in the preferred embodiments the back face and bottom face are at an angle to define a reservoir so that urine always collects or pools adjacent the outlet point, when it is removed from the device at a peak flow rate of up to typically 60 millilitres per second.

Embodiments of the invention provide a urine collection apparatus, comprising a funnel having a front wall positionable towards the pubic bone area of the user, side walls being positionable either side of the of the area of the user's body from which urine is discharged and a rear wall, positionable towards the perineum area of the user, the upper edge of the front and forward parts of the side walls forming an opening for the funnel which extends around the area of the user's body from which urine is discharged, the funnel also having an outlet by which urine can be removed from the urine collection device, wherein the opening for the funnel leads to a urine receiving area formed by the front wall and forward parts of the side walls of the urine collection device, said urine receiving area leading to a urine collection reservoir formed of the rear wall and rearward parts of the side walls of the urine collection device, said urine collection reservoir having an upper wall extending from the opening of the funnel towards the rear wall so that the urine collection reservoir defines in use with the user's body a substantially closed chamber which receives urine indirectly from the user via the urine receiving reservoir, said urine collection reservoir leading to the outlet so that urine can be removed from the funnel, the outlet being connected to a tube leading to a suction pump by which urine from the urine collection device can be removed to a separate urine collection chamber.

Preferably the funnel includes a weir to prevent or inhibit backflow of urine away from the direction of suction.

Conveniently, a valve is incorporated within the apparatus such that urine exiting from the funnel cannot flow back under gravity when the suction pump is deactivated.

Any suitable pump may be used which provides a high flow rate (e.g. up to 40 litres/minute (air)) so that it can achieve the high flow rates required to meet the initial instantaneous flow rate that occurs when a person is urinating. A suitable pump is a linear diaphragm pump.

Advantageously, the pump is self limiting in terms of negative pressure that can be produced. This provides an inherent safety feature in that it avoids the production of unsafe vacuum within the funnel which can harm the user of the urine collection device.

Advantageously, the pump includes a silencer and/or a carbon filter to reduce odours.

Typically, the suction pump leads to a collection vessel which is a sealed airtight vessel, which can be depressurised by the pump to a negative pressure of about 270 millibars.

It is preferred that the pump is controlled by a processor which can run a check program to ensure correct operation, and connection of the pump to the urine collection device and/or the separate urine collection chamber. The check program may be preceded by an indication of whether the pump is on or off and this can be provided by a visual indication on a display or an audible warning. Once the pump is judged to be operating correctly, an indication may be given to the user that they can start using the pump with the urine collection device.

Preferably, the processor runs a test program which prompts the user to check that the arrangement is operating properly after the pump is switched off. An example of this is where the pump has been switched off to empty the urine collection chamber. An indicator is activated after the chamber has been emptied to prompt the user to run the check program again. The test program is controlled as a result of receiving signals from various parts of the pump arrangement. A sensor may be associated with the container to check that it has been properly connected to tubing associated with the pump.

Further sensors may be provided, e.g. in tubing leading to and from the pump to detect the pressure at one or more points to monitor for leakages or blockages in the system.

In one test routine the pump is caused to apply suction to the flow path upstream of the pump arrangement to set a negative pressure, and a check is made to see if this pressure is reached and held for a predefined period of time. If the check result is a negative result, the user will be prompted to check the system to ensure that tubing and connections between the tubing and components such as the container for the urine are secure.

Advantageously there is a hydrophobic filter membrane between the collection vessel and the pump to prevent any potential ingress of urine into the pump, which could otherwise have major health and safety implications. Further, the filter membrane assists in preventing ingress of bacteria.

It is envisaged that the collecting vessel may be calibrated to measure the amount of urine the user is releasing so that flow rates of fluid through the body can be monitored.

Advantageously, the collection chamber includes a disposable bag having a tap at the bottom for emptying urine from the bag. This allows the bag to be used for a given a number of times, much like catheter bags, which are periodically drained. In a preferred arrangement, the collection chamber is associated with a sensor which can provide a signal for an indicator to inform a user when the bag within the collection chamber is full or has reached a certain fill limit and therefore needs emptying. In one exemplary routine, the system sounds an audible alarm after completion of the current “void” or urination. The processor also prevents the pump from being operated again until a reset operation has been carried out. Further, the collection chamber may have a sensor that can monitor the flow rate of urine over a particular period of time to assess whether a user is producing urine at an appropriate amount relative to their fluid intake. By measuring flow rates, conditions such as oedema or urinary blockages may be detected.

In a preferred arrangement, the pump is provided as a portable device. It is envisaged that when the pump is portable, said pump is provided as a separate unit attachable to a docking station that can also accommodate the urine collection chamber.

It is envisaged that the docking station may be connectable to an external power source such as the mains electricity supply. However the pump may include its own integral power supply such as a rechargeable battery pack.

Embodiments of the invention also provide a urine collection apparatus comprising a funnel for receiving urine, a pump arrangement for pumping urine passing into the funnel, and a collection device for receiving collected urine, said funnel having an outlet which is connected to tubing leading to the pump arrangement, wherein the pump arrangement includes sensors to detect one or more parameters concerning itself, the tubing and/or the collection device, and indicators to alert a user of the device whether defined parameters fall within or outside said defined parameters.

It is preferred that the pump arrangement includes a check program to monitor pressure within said urine collection arrangement.

Advantageously, a test program is provided with an indicator that prompts the user to check that the arrangement is switched on. Preferably, as well as checking whether the arrangement is switched on, the user is prompted to check that all components are present and connected properly. The check may comprise visual indicators alerting the user of the stages in the check process to be taken. Alternatively, an audible prompt may be used to talk the user through the various stages in the check procedure.

Preferably, the test program is operable to detect when the collection chamber been emptied and to alert, the user to run the check program again. The test program may be controlled as a result of receiving signals from various parts of the pump arrangement. A sensor may be associated with the container to check that it has been properly connected to tubing associated with the pump.

Further tubing can also include sensors at one or more points to receive information about blockages or power leaks in the pump system.

Preferably, the pump is provided as a portable device, which can be docked with a base station adapted to receive said pump and a urine collection chamber.

It is envisaged that the pump includes its own power supply or it can be connected to a power supply provided by the base station or from mains electricity.

Embodiments of the invention also provide a portable urine collection system for use with the urine collection devices described, the system including a system housing containing an electrically operable suction pump, a sealable container in or on the housing for containing a urine collection bag, the container having a lid and being connectable via tubing to a urine collection device, the container also being connectable to said pump such that when the lid is closed and the pump is activated air or fluid is drawn into the urine collection device and, via the urine collection bag container, to the pump, to thereby provide suction permitting urine to be drawn into the urine collection bag when the urine collection device is being used.

Conveniently, the housing is adapted to be releasably secured to an item of furniture such as a hospital bed and includes on an upper surface a beaker or drip tray and associated beaker housing, the housing for the beaker allowing the beaker to be orientated in respectively opposite positions, the inside of the drip tray being shaped to permit the open end of the collection device to be releasably stored therein only in one of said orientations, thereby permitting either hand of e.g. a user in the bed to pick up the urine collection device in its correct orientation, depending upon which side of the bed the system housing has been secured to and which way the beaker is oriented.

The invention will now be described, by way of example only, with reference to the accompanying drawing figures in which:

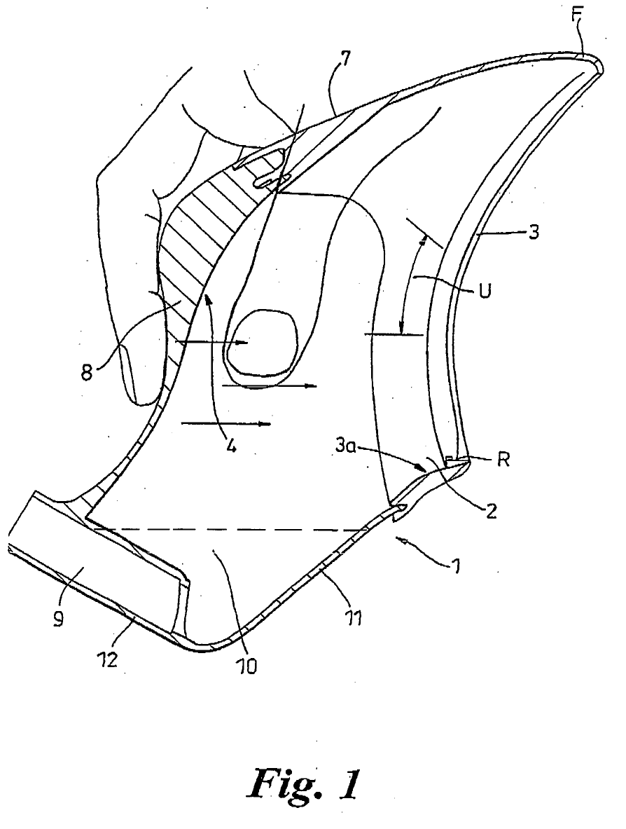

FIG. 1 shows in section a first embodiment of a urine collection device according to the invention,

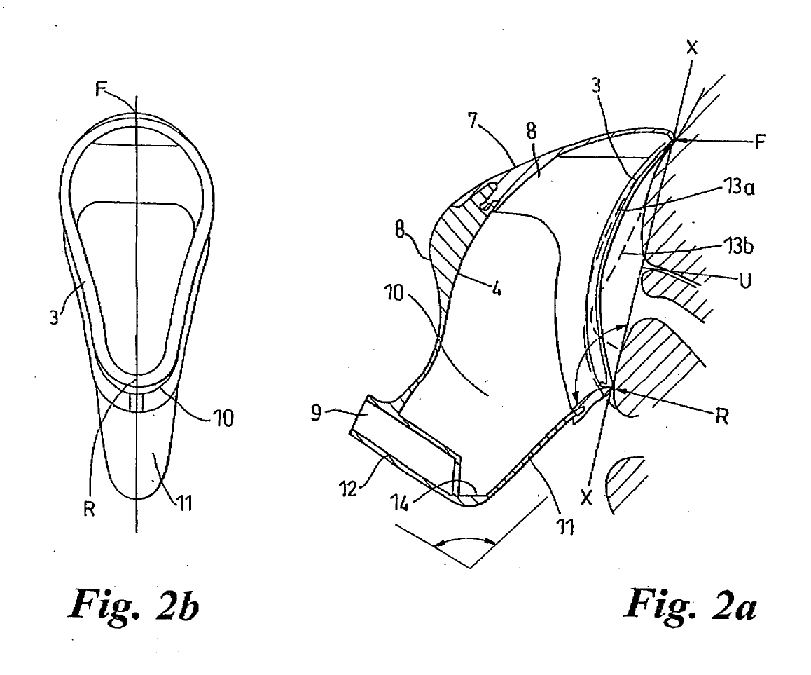

FIG. 2a shows a urine collection device of FIG. 1 in situ against the body of a user,

FIG. 2b shows a front perspective view of the urine collection device of FIG. 1,

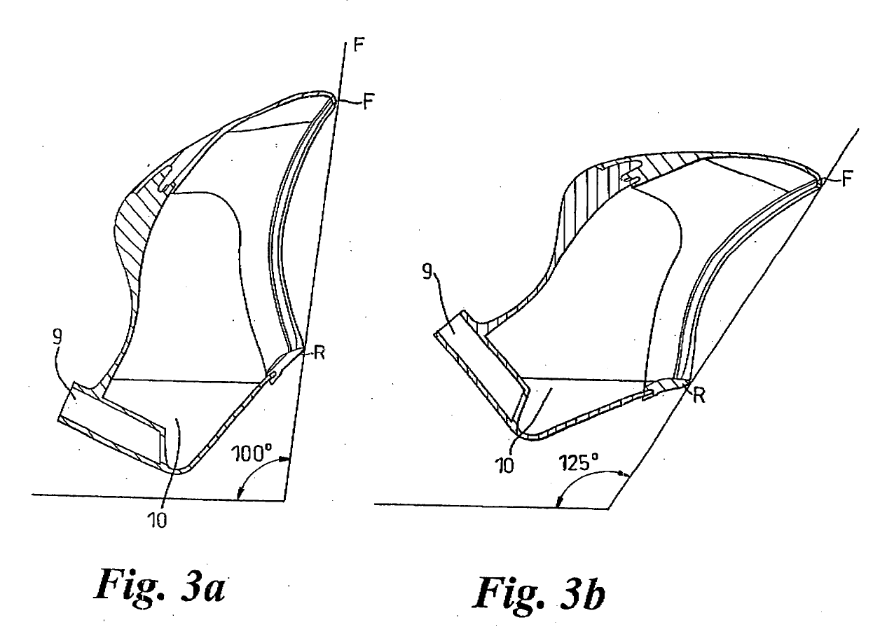

FIG. 3a shows the tilt angle of a urine collection device according to a first aspect, when a user is in a reclined sitting position,

FIG. 3b shows the tilt angle of a urine collection device according to a first aspect, when a user is in a supine position,

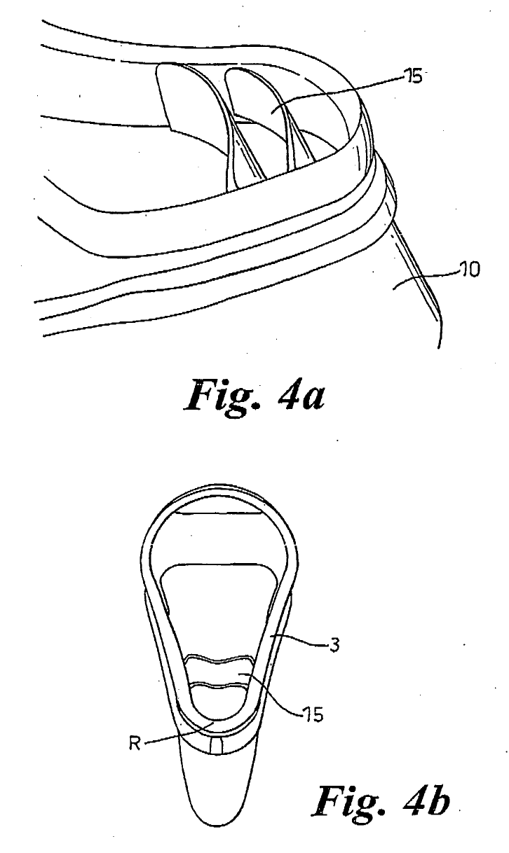

FIG. 4a shows additional weirs positioned on the inside part of a urine collection device according to a second embodiment of the invention,

FIG. 4b shows a view from above of the second embodiment of the urine collection device of FIG. 4a including weirs,

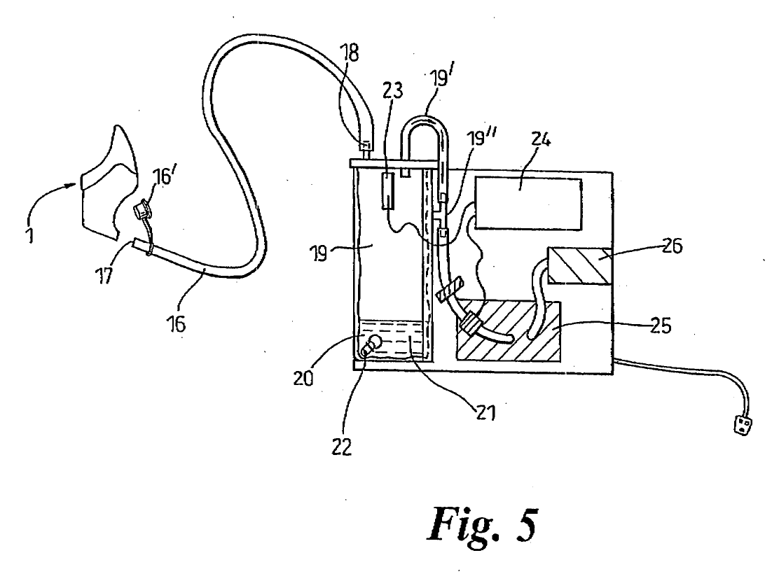

FIG. 5 shows a suction pump and associated apparatus to be used with a urine collection device of this invention,

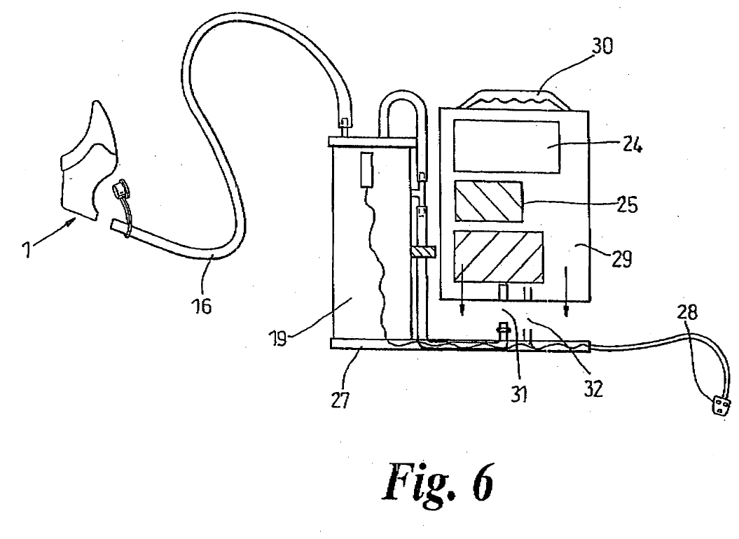

FIG. 6 shows a portable pump and associated apparatus to be used with a urine collection device according to another embodiment of the invention,

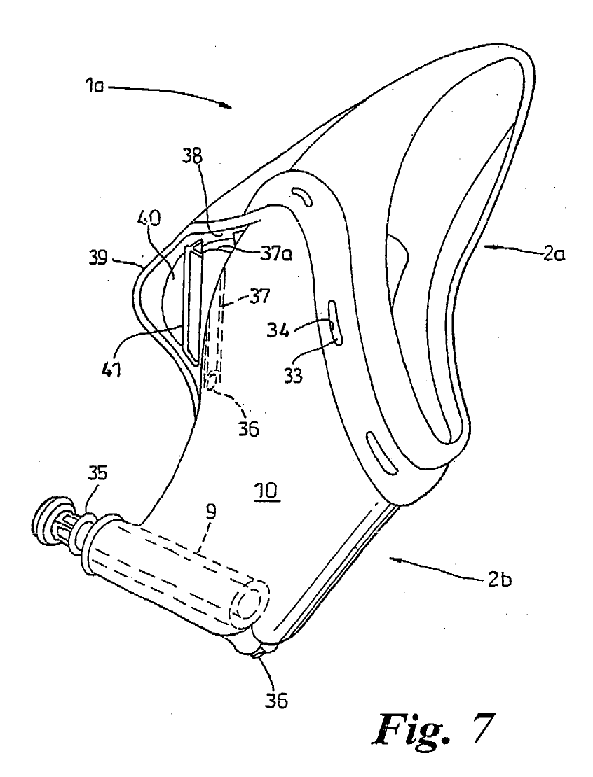

FIG. 7 shows a third embodiment of urine collection device,

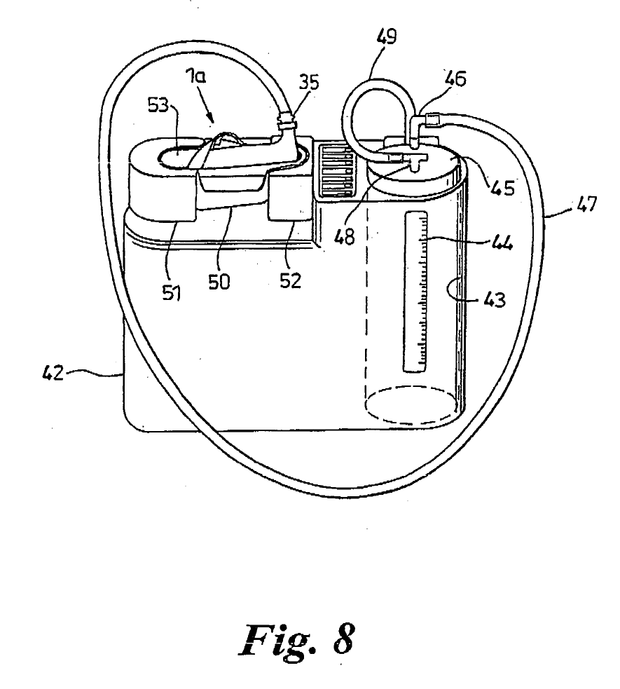

FIG. 8 shows a portable urine collection system incorporating the urine collection device of FIG. 7,

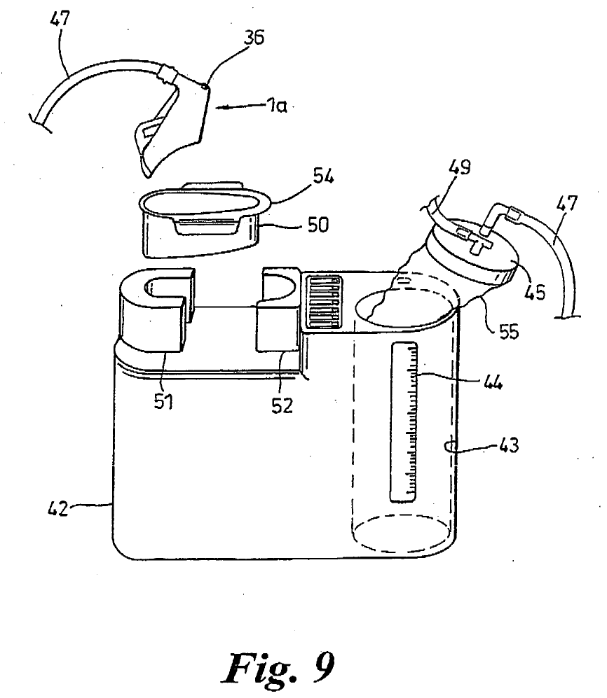

FIG. 9 is an exploded view of the system shown in FIG. 8,

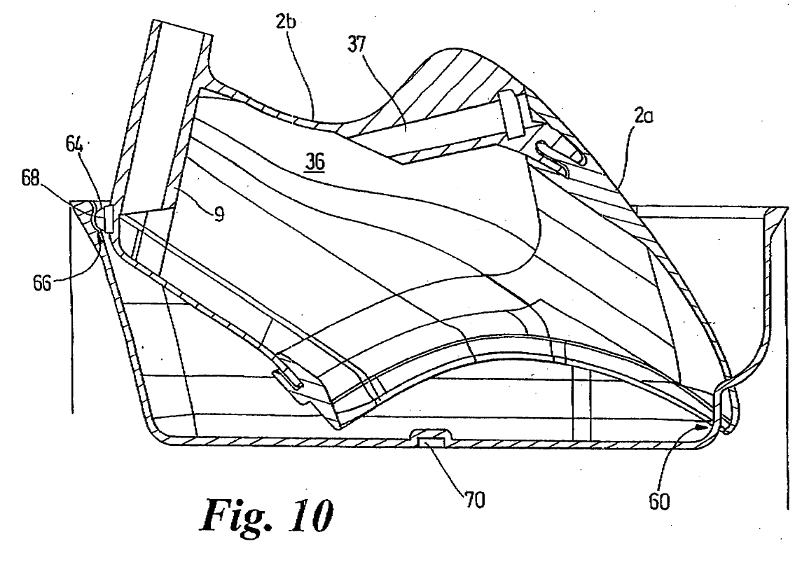

FIG. 10 is a side section view of the urine collection apparatus of the system of FIGS. 8 and 9 retained within its beaker, and

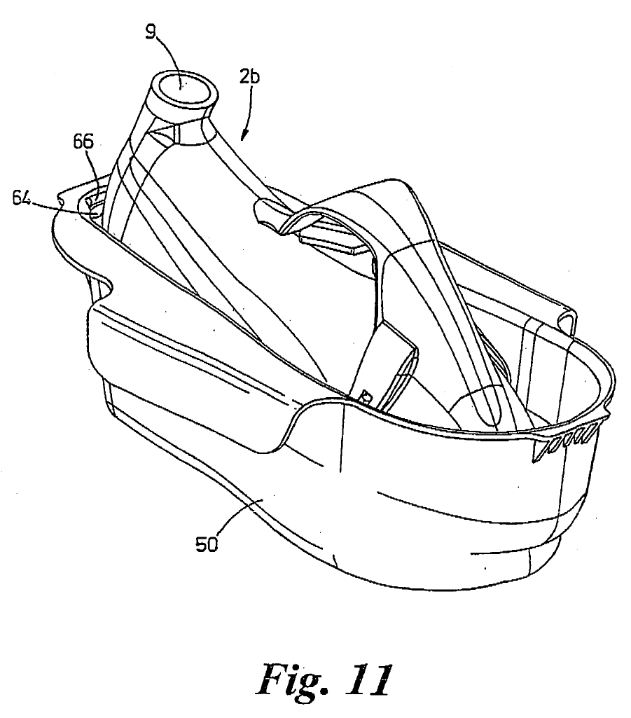

FIG. 11 is a perspective view of the components shown in FIG. 10.

Referring firstly to the embodiments of FIGS. 1 to 3, the urine collection device shown generally at 1 comprises a funnel body 2 having an open rim 3 defining a urine inlet opening for the device 1. Upper rim 3 has an upper contact region F which contacts the mons pubis or pubic bone area of a user of the device, and a lower contact region R, which contacts the perineum area of the user. Between the upper contact region F and lower contact region R are the side walls whose rim contacts the labia majora, and which is concavely curved when seen in side view (FIGS. 1 and 2a). The mouth of the funnel in use is urged against the user's body to form an enclosed chamber which receives urine discharged from the user's urethra.

The funnel body 2 has a smoothly contoured, generally concave, inner front face 4. The funnel body 2 has a contoured outer front face 7 leading from contact point F to form a bowed concave area 8, which is the typical region of where a user holds the urine collection device. Internally, the area forms the urine receiving area of the urine collection device and is the part of the device where the urine directly enters the device via the opening 3. The front curved area 7 leading to 8 forms a contoured ‘S’ curve leading towards outlet tube 9. The outlet tube 9 leads from an extension of the urine collection device 1 which extends forwardly of the opening 3 to form the extension that provides a urine collection reservoir 10 or basin for the device. This urine collection reservoir or basin is provided as a chamber that collects urine prior to it passing to outlet tube 9. The chamber includes a back wall 11 and bottom wall 12, which are angled with respect to one another to define the collection chamber 10. Area 3a is the upper surface of a weir which has a rearwardly directed edge designed to seal in to the perineum. The periphery of the rim 3 apart from the area 3a is formed with an inturned lip which acts as a seal and as a gutter which drains onto the weir surface.

FIG. 2a shows urine collection device 1 in situ against a user of the device. The urethra of the user is shown as U and the front and rear contact points F, R are where the urine collection device contacts with the user's body (the mons pubis and the perineum respectively). Edge 3 will also contact with the body. The labia majora 13a will seat under the walls of the opening 3, forming a seal around the top of the opening. The labia minora 13b will be positioned within the device and can protrude up to about 18 mm within the device, the labia minora 13b being located well within the opening 3. The contact points F and R provide a medial axis x-x relative to the user's body.

The back wall 11 is steeply angled relative to the axis x-x. Typically this angle is about 146° but as mentioned the angle may be 130-160°. If the angle is much greater, then the back wall 11 of the collection device 1 would press against the buttocks of the user making it difficult or impossible for the lower contact region R to seal properly against the user's perineum. Also, for users in a reclined position, there must be sufficient clearance between the lowermost point of the device and the surface on which they are lying. The back wall 11 is also angled relative to bottom wall 12 and the typical angle between these walls is about 106° with a range being 90-120°. By this arrangement urine is caused to pool in the collection reservoir 10 of the urine collection device, whether the user is sitting or lying down. Urine is directed from the urine receiving area formed by curved face 4 into the urine collection reservoir 10. The outlet tube 9 is provided with its opening located in the lowermost part or trough of the reservoir so that the reservoir can be completely drained via the tube 9. FIG. 2b is a perspective view from above of the urine collection device of FIGS. 1 and 2 showing the opening 3 front part F rear part R and urine collection reservoir 10.

It can be seen that the urine collection reservoir is offset from opening 3, rather than being positioned directly under it.

FIGS. 3a and 3b respectively show the position of the urine collection device when a user is in a reclined sitting position, which is usually when a user is propped up in bed by several pillows, and when a user is lying down in the supine position. As shown in FIG. 3a, when a user is reclining, typically the device is held with the axis running through contact points F and R disposed at about 100° to the horizontal.

FIG. 3b shows the device when it would be used when a user is in the supine position, when typically, the corresponding angle to the horizontal is about 125°, which is a much less steep angle than when the user is reclining. However, from both positions, it can be seen that the urine collection reservoir 10 is always maintained as the lowest part of the device and it is here that urine is held prior to it being removed through opening 9a of outlet pipe 9.

FIG. 4a is a cut away view of the rear part of a modified embodiment of the urine collecting device having vanes 15 on the internal surface of the device. These vanes provide weirs which are angled such that urine would be directed towards collection point 10 and ultimately to outlet 9. These vanes are designed to catch any portion of urine running down the user's body inside the device and to direct it towards the urine reservoir 10 before it reaches the lower portion R of the rim.

FIG. 4b shows the view from above of the vanes 15 relative to opening 3.

FIG. 5 shows an embodiment in which a pump 25 is used with a urine collection device of the invention. This pump may be used with a urine collection device as previously described or with other types of urine collection devices. The pump is a high airflow linear diaphragm pump with a free airflow of up to 40 litres per minute.

A urine collection device 1 is attached to a tube 16, having an inlet 17 for attachment to the urine collection device 1, on the end of which tube 16 may be a cap 16′ to close off the tube if needs be. The tube is preferably 8 millimetres in internal diameter and a typical length of tube is around 1.8 metres. The other end of the tube 16 is attached to an inlet 18 to a collecting vessel 19. Typically the collection vessel 19 is under negative pressure by means of a suction tube 19′ passing to the suction pump 25. The collecting vessel 19 may include a disposable bag 20, which stores urine 21, and the disposable bag can have a outlet valve 22 for draining off urine when it gets to a particular level. The collecting vessel 19 is also exposed to the suction from the pump by a T-piece connector 19″, which balances the pressure inside and outside the disposable bag 20. The level of urine in the bag 20 is detected by a sensor 23 which typically is a level sensor located adjacent to the collecting vessel and senses when the bag fluid level has reached a predefined level and which signals this to a controller 24 which operates a cut off switch which will not allow the pump to restart until the bag 20 has been emptied. The controller 24 can also give an audible and/or visual alarm when the bag needs to be emptied. As indicated above, the controller preferably notes when the level sensor 23 is tripped, allows the present urine collection cycle to finish, but then flags by e.g. a flashing light, that the bag needs changing, and further prevents start of a new urine collection cycle until the sensor 23 indicates that the bag has been changed, and the user has performed a reset operation. The controller can include other lights to indicate ON/OFF as well as an indicator to show that the pump 25 is running at required parameters. The controller may also include a prompt for a test programme to be run. It is envisaged that if a test programme is not run successfully, the pump cannot be used. The test programme tests that the bag has been changed and that there are no air leaks in the system or blockages which could affect the successful use of the equipment by the user. The test programme also carries out a system pressure test by evacuating the system to set a negative pressure, and checking that this pressure is reached and maintained. In order to do this, the operator must close a clamp on the tube (e.g. using the cap 16′) and then press the test programme button. If the test is successful, the controller 24 will display this. The controller is also programmed to turn on the pump as soon as the urine collection device is removed from its beaker on the system, and to turn the pump off a set time after the collection device has been returned to the cradle after a urine collection cycle, to purge the system and collect drips etc in the collector device or tubing.

In order to reduce odours etc, there is a charcoal filter 26 which may be incorporated or provided separately from a silencer. Such arrangements make the device more pleasant to use in that it reduces odour and maintains a quiet environment when the pump is being used.

The arrangement shown in FIG. 5 is a one-to-one arrangement where a mains powered single pump is used with an individual urine collection device. FIG. 6 shows a portable urine collection device in accordance with another embodiment of the invention. As before, a tube 16′ is attachable to a urine collection device 1, and a collection chamber 19, and is attached to a base 27. This arrangement is connected to an electrical power source by an electric plug 28. This base provides a base station onto which a portable pump arrangement 29 can be seated. This arrangement has a pump 25 and a controller 24 as previously and a carry handle 30. The pump arrangement 29 is docked onto the base station and connections are made with tubing for air to be sucked through via the pump 25. The power supply between the base station and the portable pump is provided by electric connector point 32. As before, tests will be made using the indicator panel 24 to ensure that appropriate electrical connections have been made and that urine has been correctly connected so that the device will operate properly.

By providing a portable pump device 29 that may be transmitted between users, there is a cost consideration that one pump can be provided by e.g. several users on a ward. The portable pump/control 29 unit can also contain a rechargeable battery pack so that the device does not necessarily have to be plugged into the main power supply. Further, by having parts that can be interconnected, some parts may be provided as disposable elements that can be replaced easily while other parts of the arrangement can be dismantled for sterilization when needs be.

Referring now to FIG. 7, there is shown a perspective view of an alternative embodiment of urine collection device 1a to that shown with reference to FIGS. 1 to 4 although it is of the same general internal shape. In this embodiment the funnel body is in two part form, an upper, resilient, part shown generally at 2a and a lower generally rigid, part shown at 2b. The upper part 2a is typically made of an elastomeric material, and the lower part 2b typically being made of rigid plastics. With this arrangement, spaced raised formations 33 disposed circumferentially around the upper rim of the lower part 2b, co-operate with correspondingly shaped apertures 34 provided on the lower rim of the upper part 2a, to connect the upper part 2a detachably to the lower part 2b. This allows the upper part 2a to be replaced with either a new one, or even one of a different size and shape, and similarly the lower part 2b may be replaced as required.

In this embodiment of urine collection device, the urine exit tube 9, shown partially in broken outline, leads to an annular connector spigot 35 onto which may be push fitted the socket end of an adaptor fitted to the end of a flexible tube for removing urine from the urine collection device 1 in a manner to be described.

On the lower outside front wall of the lower part 2b of the funnel is mounted a small magnet 36 which can be used for indicating the presence of the funnel 1 to the controller, again in a manner to be described.

Above the tube 9 in a generally upright orientation is an aperture 36, shown in broken outline forming one end of a generally upright tube 37, shown partially in outline leading from the inside of the funnel to the outside immediately below a roof portion 38 of a finger handle 39, having a central web 40. This has the advantage in that air can be drawn into the urine collection device and delivered to a point just above the highest expected liquid level in the urine collection reservoir, without the stream of urine affecting the flow of air and without, of course, the possibility of any of the urine escaping from the device via the tube 37. In order to protect the open, upper, end of the tube 37 from accidentally being blocked by a finger of a user of the device, a pair of planar finger guards 41 (only one of which is shown) are positioned on either side of the inlet tube 37 so that its inlet end 37a cannot be accidentally blocked from the ingress of air.

Referring now to FIGS. 8 and 9, there is shown a front perspective view of a complete portable urine collection system adapted to be releasably secured by e.g. hook formations, onto the side or end of a hospital bed. The system comprises a housing 42 containing a vacuum pump (not shown) and also a urine collection bag container 43, shown partially in broken outline, having a window 44 for visually inspecting via a suitable scale the level of liquid within a urine collection bag 55 (shown in FIG. 9). The top end of the urine collection bag container 43 is closed by means of a lid 45 through which extends an elbow-shaped hollow spigot 46 onto the end of which is push fitted the end of a flexible urine collection tube 47, connected at its other end to the spigot 35 of the urine collection device 1a of FIG. 7.

Also extending through the lid 45 is another elbow 48 through which air can flow from the inside of the container 43 to an outlet tube 49 connected at one end to the outlet end of the elbow 48 and at its other to the inlet end of a conduit (not shown) on the outside of the housing 42 leading to the pump, to thereby permit air to be drawn through the elbow 48 and outlet tube 49 into the pump.

The urine collection apparatus 1a is removably located within a beaker 50 of symmetrical outer shape such that it can be received within correspondingly shaped cradle members 51, 52 in either of two different orientations, at 180° to each other. The inside surface 53 is, however, shaped to closely follow the outer contours of the urine collection device 1a so that the latter may be oriented in the beaker 50 in only one position. The two possible orientations of the beaker mean that the apparatus can be set up to present the beaker (and the urine collection apparatus 1a) in the appropriate orientation for use according to whether the device is on the left or right side of the user. It is important for intuitive operation that the urine collection apparatus 1a is always oriented so that the funnel opening is facing rearwardly, so that the user does not need to rotate the funnel when transferring it from the beaker to their body. Equally it is important that the urine collection apparatus is always returned to the beaker in the correct orientation.

As will be seen more clearly with reference to the exploded view of FIG. 9, the beaker 50 has a flanged upper edge 54 which extends around its entire periphery so that when the beaker 50 is received between the cradles 51, 52 it may be orientated in either of two opposite directions allowing the system to be set up with the urine collection apparatus in the required orientation, thereby permitting use of the apparatus on either side of e.g. a hospital bed. In turn, when not in use, the urine collection device 1a may simply be retained within the interior of the beaker 50.

The cap 45 is connected to a urine collection bag 55, which may be reusable or disposable. The beaker 50 and urine collection apparatus 1a are designed so that the beaker collects any drips from the urine collection device which drip off when the latter is inverted and replaced in the beaker. The beaker and urine collection device may be removed as a single unit and taken to the sluice for washing.

Referring now to FIGS. 10 and 11, the urine collection apparatus 1a is dimensioned relative to the beaker 50 both such that the urine collection apparatus can only fit into the beaker in one orientation but also so that a unique action is required securely to locate the urine collection device in the beaker for storage. The beaker is provided in its lower portion with an inner clip surface 60 which retains the upper rim 62 of the upper part 2a. A lug 64 is formed on the lower part 2b which co-operates with a dimple 66 in an upper part of the wall at the opposite end of the beaker 50. As is seen in FIG. 10, the dimension between the lug 64 and the upper rim 62 is greater than that between the dimple and the clip surface 60. In order to clip the urine collection apparatus 1a into the beaker 50, the upper rim 62 must first be brought into engagement with the lower part of the clip surface 60, and the apparatus then swung to bring the lug 64 to clip into the dimple 66 against a bias provided by deformation of the upper part 2a, so that it clicks home.

The urine collection apparatus 1a and the beaker 50 include respective small magnets 68, 70 which co-operate with Hall-effect sensors on the housing 42 to signal the presence of the urine collection apparatus and the beaker to the control system. The magnet on the beaker 50 is located centrally and the housing 42 has a single beaker-detecting Hall-effect sensor (not shown) which detects presence of the beaker in either orientation of the beaker. Two separate funnel-detecting Hall effect sensors (not shown) are provided one in each cradle member 51,52 at opposite ends of the cradle.

The Hall-effect sensors are used by the system controller to detect the location of the urine collector apparatus 1a and the beaker 50. If just the urine collection apparatus 1a has been removed, and the beaker is still in position, the controller starts the pump ready for urine collection, and stops the pump, say, 60 seconds after replacement of the urine collection apparatus. If the controller detects that both the urine collection device and the beaker have been removed, the pump is kept inactive.

In the above embodiments it is envisaged that the funnel will only be applied to the user when they wish to urinate, and clearly the user must have bladder control. In other embodiments however the funnel may be applied to the user and held in position with e.g. a suitable belt, harness or garment for prolonged periods, with the funnel having associated therewith a sensor for detecting urine therein, and for signally a controller automatically to start operation of the suction pump, so that the device may be used by users with little or no bladder control.

Also, whilst described with female users in mind, the device may also be used with little or no adaptation for male users.

1-18. (canceled)

19. A urine collection device (1) for attachment to a pump (25) and for a user in a supine, or reclined position, said device including:
an open-mouthed container (2) having a rim (3) around the mouth for being urged in use to seat generally around the periphery of the urine discharge region to receive urine discharged from the urethra,
the container (2) being adapted to fit between the legs of a user in a supine or reclined position,
the container (2) having at its lower part a reservoir basin (10) in which fluid may collect in use when the container (2) is applied to a user in a supine or reclined position, and
an outlet (9) in the lower part of the reservoir basin (10) through which urine may be drawn from the container, characterised in that the rim (3) has a perineal wall portion (12) adapted in use to engage edgewise with the perineum to effect a fluid tight seal, and in that the container is provided with a vent (36) above the liquid level in the reservoir basin (10) in use.

20. A urine collection device according to claim 19 for a female user, wherein said rim (3) is generally of oblate curvilinear form, wherein a broader, upper end region of the rim (3) is adapted to be seated against the mons pubis (pubic bone region), and a narrower lower end region of the rim is adapted to be seated against the perineum, in use.

21. A urine collection device according to claim 20, wherein the inner surface region of the container extending inwardly from the lower end region of the rim defines a downwardly-inclined weir surface.

22. A urine collection device according to claim 19, wherein the upper and lower ends of the rim (3) define a notional contact plane (XX) which in use lies at between about 100° to 135° to the horizontal, when the user is reclined or supine.

23. A urine collection device according to claim 19, wherein the reservoir basin (10) is defined at least partly by a back wall portion (11) and a bottom wall portion (12) which converge downwardly towards said outlet (9).

24. A urine collection device according to claim 23, wherein the reservoir basin (10) has a capacity of at least 10 ml and preferably at least 20 ml, when the device is applied to a user in a supine position.

25. A urine collection device according to claim 23, wherein said back wall portion (11) and said bottom wall portion (10) converge at an angle of between 90° and 120°, and preferably at about 106°.

26. A urine collection device according to claim 23 when dependent on claim 4, wherein said back wall portion (11) lies at an angle of between about 120° and 160°, and preferably about 146° to the notional contact plane.

27. A urine collection device according to claim 19, which further includes in the lower region of the mouth at least one weir plate means (15) which extends between opposed portions of the rim (3) and extending into the basin region (10) of the container.

28. A urine collection device according to claim 19 wherein the outlet includes an tubular portion (9) integral with the container and having a drain inlet end which is located at the lowermost portion of the reservoir basin (10), and which extends upwardly to terminate in a drain outlet end.

29. A urine collection device according to claim 19, wherein the rim (3) has an inturned resiliently deformable lip extending around a part thereof for seating against the user's body and providing an internal peripheral gutter.

30. A urine collection device according to claim 19, wherein said vent (36) includes a breather tube (37) disposed generally vertically, the upper end of said tube being located to the exterior of said container, to prevent escape of urine.

31. A urine collection device according to claim 30, wherein said container is provided with guard surfaces (41) to prevent inadvertent blocking of the upper end of said tube (37) by the hand of the user in use.

32. A urine collection system, comprising a urine collection device (1) according to claim 19, a suction pump (25) for applying suction to said device, and a container (43) for receiving urine drawn from said collection device (1) by said suction pump.

33. A urine collection system according to claim 32, wherein said urine collection device (1) has associated therewith a urine sensor and said system is operable on detection of urine by said sensor to start said suction pump (25).

34. A urine collection system according to claim 32 wherein said vent (36) is sized such that, in use, when the urine collection device (1) is applied to the user, and the suction pump (25) operated, a slight negative pressure is present in the inside of the device to aid location and sealing of the device against the user.

35. A urine collection device (1) for attachment to a pump (25) and for a user in a supine, or reclined position, said device including:
an open-mouthed container (2) having a rim (3) around the mouth for being urged in use to seat generally around the periphery of the urine discharge region to receive urine discharged from the urethra,
the container (2) being adapted to fit between the legs of a user in a supine or reclined position,
the container (2) having at its lower part a reservoir basin (10) in which fluid may collect in use when the container (2) is applied to a user in a supine or reclined position, and
an outlet (9) in the lower part of the reservoir basin (10) through which urine may be drawn from the container, characterised in that said reservoir is defined at least partly by a back wall portion (11) and a bottom wall portion (12) which converge towards said outlet (9), and in that said back wall portion (11) lies at an angle of between 90° and 120° to a notional contact plane (XX) defined by the upper and lower ends of said rim (3).
